(19) United States
(12) Patent Application Publication
(10) Pub. No.: US 2003/0223943 A1
(43) Pub. Date: Dec. 4, 2003

(54) Transparent cosmetic lipsticks

(76) Inventors: Yuh-Jye Uang, Chesterfield, MO (US); Cheng-Teh Lin, Taipei (TW)

Correspondence Address: Paul M. Denk, 763 S. New Ballas Road, St. Louis, MO, 63141, US

(21) Appl. No.: 10/439,835

(22) Filed: May 16, 2003

Related U.S. Application Data
(60) Provisional application No. 60/383,920, filed on May 29, 2002.

(51) Int. Cl. A61K 7/025

(52) U.S. Cl. 424/64

(57) ABSTRACT

The present invention is provided a lipstick that are stable, solid, good transparency, and good wear properties. The transparent lipstick comprises: about 15% to about 70% by weight of gellant, about 20% to about 55% by weight of solvent, about 1% to about 35% solubilizer

### CROSS-REFERENCE TO RELATED APPLICATION

This application is a non-provisional patent application based upon provisional patent application having Serial No. 60/383920, filed on May 29, 2002, which is owned by the same inventor.

### TECHNICAL FIELD OF THE INVENTION

The invention relates to transparent cosmetic sticks, preferably lipsticks, which are free of sweating and syneresis.

### BACKGROUND OF THE INVENTION

In general, the clear or transparent cosmetic products are particularly attractive because consumer likes transparency to quality and purity. In accordance with these principles, there are other clear consumer products such as clear gels for antiperspirants, deodorants sticks, and gel toothpastes. Traditionally compositions used to make transparent lipstick have possessed one or more undesirable characteristics. Most transparent lipsticks do not posses enough rigidity in its composition and need to be enclosed in some type of supporting container. Some lack hardness while other has succumbed to the problems of sweating and syneresis. Thus the desired for a transparent lipstick that has structural integrity and storage stability will certainly prove to having a higher consumer demand than previous products of clear lipsticks.

U.S. Pat. No. 3,148,125 to Strianse et al. discloses a clear lipstick requiring medium molecular weight of polyamide, fatty acid ester, fatty alcohol, mineral oil, and lower alcohol such as ethanol, isopropyl alcohol. By using lower alcohol, which is known to cause irritation and is undesirable in its application for use on areas having mucus membranes. The medium polyamide will also cause the sweat and syneresis.

U.S. Pat. No. 5,610,199 and U.S. Pat. No. 5,780,517 to Cohen et al. discloses a transparent lipstick comprising a dibenzyl monosorbitol acetate (DBMSA), lipophilic materials and one or more cosmetic materials. U.S. Pat. No. 5,750,125 to Lahanas et al. disclose a clear lipstick with color comprise dibenzyl monosorbitol acetal(DBMSA) and a refractory material having a refractive index of between about 1.50 and about 1.65. Japan Pat. No. 63,119,405 to Yokota Takashi discloses a transparent lipstick containing 12-hydroxystearic acid, a polyhydric alcohol ester of rosin and polyhydric alcohol partial ester of a side-chain fatty acid or unsaturated fatty acid, and solvent Japan Pat. No. 4091010 to Kato Toru discloses the transparent solid cosmetic obtained by containing 12-hydroxystearic acid, transparent liquid oily ingredient such as squalane and methylphenylpolysiloxane. Japan Pat. No. 2000-204,012 to Kummel Takayuki discloses the transparent lipstick comprises 12-hydroxystearic acid, silyl containing polysaccharide, oily substance such as vegetable oil or Vaseline and trimethyl silylated silicic anhydride. The previously mentioned patents, all the transparent cosmetic formulas have comprised three type of gallants, which are medium polyamide, DBMSA and 12-hydroxystearic acid. In general, the concentration of either DBMSA or 12-hydroxystearic acid in a particular composition influences the hardness and clarity of the composition. An increase in the concentration of either DBMSA or 12-hydroxystearic acid will provide a harder, less transparent composition. Conversely, a decrease in the concentration either DBMSA or 12-hydroxystearic acid will provide a softer, more transparent composition.

### SUMMARY OF THE INVENTION

The present invention is directed towards transparent cosmetic stick comprising of one or more polyamide gellants selected from the group consisting of fatty polyamide and ester terminated polyamides. One or more solvent, wherein one of the solvents is a fatty acid ester having a carbon chain of between about 9 to about 30 carbons. One or more solubilizers, wherein one of the solubilizers is a fatty acid having a carbon chain of between about 1 to about 22 carbons. The solubilizer comprises at least 1% by weight of the composition.

It is an object of the present invention to provide such lipstick has an even coverage, high shine, good transparency, and good wear properties.

It is another object of the present invention to provide such a lipstick that is not sticky to the touch, has good chemical and physical stability and adhesion to the lip

Further objects and advantages of the subject invention will be apparent to one of ordinary skill in the art or will be pointed out hereinafter.

### DETAILED DESCRIPTION OF THE INVENTION

As used herein “composition” refers to a base material from which solid transparent lipstick of the present invention can be made. The composition includes a gellant, a solvent and a solubilizers, all mixed together. The gellant is the component that provides structure to the composition; however, the properties of that structure may be affected by many factors, such as the amount of solvent used and the type and amount of other additives including oils. The solvent binds to the gellant and the solubilizer assists in this coupling; the combination of the gellant, the solvent and solubilizer provides the lipstick with many of its desirable properties, including clarity and storage stability.

As used herein, a fatty acid is composed of a chain of alkyl groups containing from 1 to 24 carbon atoms(usually an even number) and characterized by a terminal carboxyl group —COOH. A fatty acid is a carboxylic acid derived from or contained in an animal or vegetable oils. Fatty acids may be saturated or unsaturated. A saturated fatty acid is a fatty acid in which the carbon atom of the alkyl chain is connected single bonds. Examples of the saturated fatty acids include, but are not limited to, butyric (C4), lauric acid(C12), Palmitic acid (C16) and stearic acid (C18). An unsaturated fatty acid is a fatty acid in which there are one or more double bonds between the carbon atoms in the alkyl chain. Examples of unsaturated fatty acids include, but not limited to oleic acid (C18), linoleic (C18), and linolenic (C18).

Unless otherwise indicated, a fatty acid ester is a fatty acid with terminal carboxyl group —COOH, replaced by the alkyl group of a monohydric alcohol. R′OH, yields the fatty acid ester RCOOR′. R′, the monohydric alcohol, is composed of a chain of alkyl groups containing from 1 to 22 carbon atoms and may be saturated or unsaturated. Thus, R, the fatty portion of the fatty acid ester, is composed of a chain of alkyl groups containing from 4 to 22 carbon atoms and may be saturated or unsaturated. Examples of the saturated fatty acid esters include, but are not limited to, butyric (C4), lauric acid(C12), Palmitic acid (C16) and stearic acid (C18). An unsaturated fatty acid ester is a fatty acid ester in which there are one or more double bonds between the carbon atoms in the alkyl chain. Examples of unsaturated fatty acids include, but not limited to oleic acid (C18), linoleic (C18), and linolenic (C18)

Unless otherwise indicated, a polyol fatty acid ester is a fatty acid with terminal carboxyl group —COOH, replaced by the alkyl group of a polyfunctional alcohol yields the polyol fatty acid ester (RCOO)₃R′. R′, the polyol alcohol, is composed of a chain of alkyl groups containing from 3 to 6 carbon atoms. Thus, R, the fatty portion of the fatty acid ester, is composed of a chain of alkyl groups containing from 6 to 22 carbon atoms and may be saturated or unsaturated. Examples of the saturated fatty acid esters include, but are not limited to, Caprylic acid (C8), Capric acid (C10), lauric acid(C12), Palmitic acid (C16) and stearic acid (C18). An unsaturated fatty acid ester is a fatty acid ester in which there are one or more double bonds between the carbon atoms in the alkyl chain. Examples of unsaturated fatty acids include, but not limited to oleic acid (C18), linoleic (C18), and linolenic (C18).

Unless otherwise indicated, a fatty alcohol is a primary alcohol (from C1 to C24) with a straight or branched alkyl chain. The carbon chain can be saturated or unsaturated. A saturated fatty alcohol is a fatty alcohol I which the carbon atoms of the alkyl chain are connected by single bonds. Examples of the saturated fatty alcohols include, but are not limited to, octyl, decyl, lauryl, myristyl, cetyl, and stearyl. An unsaturated fatty alcohol is a fatty alcohol in which there are one or more double bonds between the carbon atoms in the alkyl chain. Examples of unsaturated fatty acids include, but not limited to oleyl, linoleyl, and linolenyl.

### COMPOSITION OF THE ARTICLE

Surprisingly, it has been found that the qualities of storage stability, structural integrity, clear, self-supporting, and resilient and aesthetically desirable surface can be achieved and the limitations possessed by known transparent cosmetic stick, specifically transparent lipsticks, can be overcome through the use of a combination of a particular type of solvent, with a polyamide gellant and a particular type of solublizer. Thus, the compositon of the invention contains one or more polyamide gellants, one or more solubilizers that are fatty alcohol, fatty acid, and fatty acid ester, in combination of one or more solvents that are fatty triglyceride. The polyamide gellants are preferably fatty polyamide, ester terminated polyamides, or a mixture thereof. The solubilizers are preferably fatty acids having a carbon chain 8 to 22 atoms or fatty alcohols having a carbon chain 8 to 22 atoms, or fatty acid esters having a carbon chain 8 to 22 atoms. The solvents are preferably fatty triglyceride having a carbon chain 4 to 18 atoms. The composition may also optionally contain the additional components of hydrocarbon oil, fragrances and coloring agents.

If a fatty polyamide is used as polyamide gellant, the composition typically contains from about 3 to about 70-weight % fatty polyamide. Preferably, the composition contains from about 5 to about 50 weight % fatty polyamide. More preferably, the composition contains from about 10 to about 25 weight % fatty polyamide.

If an ester terminated polyamide is used as the polyamide gellant, the composition typically contains from about 20 to about 90 weight % of ester terminated polyamide. Preferably, the composition from contains from about 20 to about 70 weight % ester terminated polyamide. More preferably, the composition contains from about 25 to about 50-weight % ester terminated polyamide.

If a mixture of the two types of polyamide gallants is employed in the composition, the composition typically contains between about 10 to about 90 weight % total polyamide gallant. Preferably, when a mixture of the two types of polyamide gellants is employed in the composition, the composition typically contains between about 20 to about 70 weight % total polyamide gellant. More preferably when a mixture of the two types of polyamide gellants is employed in the composition, the composition typically contains between about 30 to about 60 weight % total polyamide gellant.

Irrespective of which type of solvent is used, the total amount of solvent in the composition preferably contains between about 1 to about 65 weight % solvent. More preferably, the composition contains between 5 to about 50 weight % polyol fatty acid solvent. Still more preferably, the composition contains between 10 to about 40 weight % polyol fatty acid ester solvent

The composition contains a solubilizer, which is preferably fatty acid, a fatty alcohol, a fatty acid ester or combination thereof. The composition preferably contains at least 1% solubilizer by weight. The solubilizer functions to improve the solubility of polyamide gellant in the solvent, enhancing the coupling of polyamide gellant to the solvent. The coupling assists in providing the structural integrity, clarity, storage stability and absence of sweating and syneresis properties characteristic of transparent lipstick made with the composition of the present invention. The composition typically contains from about 1 to about 35 weight % of solubilizer. The composition preferably contains from about 5 to about 25% solubilizer by weight, more preferably about 7-10% solubilizer by weight, and even more preferably 10-15% solubilizer by weight.

A hydrocarbon oil may also be included in the composition as a solvent. When employed as a solvent in the invention, the hydrocarbon oil preferably is mineral oil. It comprises from about 0% to about 70% by weight of the composition. When included, the hydrocarbon oil more preferably comprises about 3% to 15% by weight of the composition. When included, the hydrocarbon oil more preferably comprises about 5% to 10% by weight of the composition.

One or more fragrances may also be included in the composition. The total amount of such fragrances in the composition is preferably about 0-5%. More preferably between about 1-4%, and even more preferably between 2-3%.

The composition may also include one or more coloring agents. The total amount of such coloring agents in the composition is typically between about 0-5%, preferably between about 1-4%, and more preferably between 2-3%.

### COMPONENTS OF THE COMPOSITION

Solubilizer

The composition of the invention contains one or more solubilizers. Solubilization is a phenomenon that enables the formation of a solution. It is related to the presence of amphiphiles, molecules possessing the dual properties of being both polar and non-polar. In the solution that have the ability to increase the solubility of materials that are normally insoluble or only slightly soluble, in the dispersion medium. Solubilizers often have surfactant properties. Their function may be to enhance the solubility of a solute in a solution, rather than acting as a solvent, although in exceptional circumstance, a single compound may have both solubilizing and solvent characteristic. The solubilizers useful in the present invention improve the solubility of the polyamide gellant in the solvent. In enhancing the solubility of the polyamide gellant in the solvent, the solubilizer improves the clarity of the gellant/solvent blend. It is believed that the solubilizers also function as a coupling agent, coupling components in the solution and providing the solution with order and stability through physical interreactions. These physical interaction can be hydrogen bonding, polar-polar interactions, Van Der Waals interaction and related forces.

Three types of solubilzers are preferred for use with the present invention, fatty acids, fatty alcohols and fatty acid esters. The hydrocarbon chains of the fatty acid, fatty alcohol, and fatty acid ester can be linear, branched or cyclic and saturated or unsaturated. Solubilizers possessing a branched carbon chain are preferred for use in the invention. The carbon chain of the fatty acids, fatty alcohols and fatty acid esters is preferably branched in the form of an isopropyl group at the end opposite the carboxylic acid, alcohol, or ester functionality. All fatty acids, fatty alcohols, or fatty acid esters are preferably monofunctional.

Be it fatty acid, fatty alcohol, or fatty acid ester, the solubilizer used in the present invention is preferably a liquid at room temperature. If the number of carbon in the carbon chain is too high, the solubilizer becomes solid and negatively impacts the clarity of the composition, by causing turbidity. Thus, the carbon chain of the fatty alcohol is preferably between about 10 to 20 carbons. More preferably, the carbon chain is between about 14 to about 18 carbon atoms. Isostearic alcohol is an example of a preferred fatty alcohol.

The carbon chain of the fatty acid is preferably between about 6 to 22 carbons. More preferably, the carbon chain is between about 10 to about 20 carbon atoms. Even more preferably, the carbon chain is between about 14 and about 18. Isostearic acid is an example of a preferred fatty acid. Isostearic acid is a preferred solubilizer for use in the invention due to its compatibility with many of other components in the composition. When isostearic acid is used as the only solubilizer in the composition, the isostearic acid is typically present in the composition in a total amount of about 1 to 25% by weight. Preferably in an amount of about 2 to 15% by weight. More preferably, in an amount of about 5-7% by weight.

The fatty acid esters are the reaction products of C2- C22 monocarboxylic acids with C1- C22 monoalcohols. The fatty acid esters have a carbon chain length of at least about 9 carbons and preferably between about 9 to about 28 carbons. The carbon chain can be linear or branched and may be saturated or unsaturated. More preferably, the carbon chain length of these fatty acid esters is from between about 10 to about 24. Examples include, but are not limited to, fatty acid esters such as isopropyl isostearate, 2-ethylhexylstearate, 2-ethylhexylpalmitate, isopropyl stearate, isopropyl palmitate. Isopropyl palmitate is an example of a preferred fatty acid ester.

A mixture of solubilizers may be used in the invention; preferably, isostearic acid is present in this mixture. Thus, a mixture of isostearic acid with another solubilizer, either another fatty alcohol or fatty acid ester, may be used. A preferred mixture is a mixture of isostearic acid, fatty alcohol and fatty acid ester. When a mixture of solubilizers is employed, the total amount of solubilizer in the composition is typically between about 1 to 30% by weight. Preferably, the amount of total solubilizer present in the composition is between about 2 to 20% by weight. More preferably, about 3-15% by weight.

Gellant

The gellants employed in the composition of the present invention are preferably polyamide gellants. Two classes of polyamide gellants are preferred for use in the invention. The first class of polyamide gellant is based on vegetable fatty acids and Polyamines. Examples of commercial polyamides which can be used as gelling agent in the composition of the present invention are Versamid 1655® from Henkel corporation. Other commercial polyamides which can be used as the polyamide gelling agent include Uni-Rez 2620® and Uni-Rez 2626®. The Uni-Rez polyamides are by Union Camp Corporation, and the Versamid polyamides are by Henkel Corporation. The second type of polyamide gellant is based on complex fatty acids that are terminated by esters such as the UniClear series from Arizona Chemical company. Such a gellant is referred to as an ester-terminated polyamide(ETPA). This type of polyamide gellants are commercially available from Arizona Chemical Company as UniClear and Sylvaclear. Examples of such commercial ETPA gellants which can be used as the polyamide gelling agent in the composition of the present invention are UniClear 80®, UniClear 80V®, UniClear 100®, UniClear 100V® and Sylvaclear A200®

When UniClear 100® is used as the only polyamide gellant, the UniClear 100® typically totals from about 15 to about 90 weight % of the total composition. Preferably, the UniClear 100® totals from about 30 to about 70 weight % of the total composition. More preferably, the UniClear 100® totals from about 40 to about 55 weight % of the total composition.

A mixture of the fatty polyamide and ester terminated polyamide may also be employed in the invention. In a polyamide gellant mixture, the fatty polyamide gellant is typically present in a weight ratio of between about 1:11 to about 3:5, i.e., 1 part per weight fatty polyamide gellant to 11 part total gellant to about 3 part per weight fatty polyamide gellant to 5 part per weight total gellant. Preferably, the fatty polyamide gellant is present in a part per weight ratio of between about 1:9 to about 1:4, i.e., 1 part per weight fatty polyamide gellant to 9 part total gellant to about 1 part per weight fatty polyamide gellant to 4 part per weight total gellant.

In a polyamide gellant mixture, the ester terminated polyamide gellant is typically present in a weight ratio of between about 10:11 to about 2:5, i.e., 10 part per weight ester terminated polyamide gellant to 11 part total gellant to about 2 part per weight fatty polyamide gellant to 5 part per weight total gellant. Preferably, the ester terminated polyamide gellant is present in a part per weight ratio of between about 7:10 to about 1:2,

Solvent

The Polyamide gellant is combined with a solvent in the composition of the invention to form a gel. The solvent is preferably a low polarity liquid that forms a gel upon being combined with a gellant. The solvents useful in the composition of the present invention is polyol fatty acid ester. The polyol fatty acid esters are made by reacting a polyol(an alcohol having more than one alcohol functionality, a polyfunctional alcohol) with a fatty acid. A glycerol fatty acid ester is an example of a polyol fatty acid ester preferred for use in the invention. Examples of glycerol fatty acid esters include lauric acid diglycerides, oleic acid triglycerides, caprylic/capric acid triglycerides, cocoglyceride stearic acid monoglyceride, palmitic acid monoglyceride. The glycerol fatty acid esters more preferred for use in the invention are those in which the number of carbon atoms of the fatty acid is in the range 6 to 22. More preferably, the caprylic/capricacid triglyceride is an example of solvent used in the invention.

The sorbitol fatty acid esters are derivatives of sorbitan. Examples include sorbitan laurate, sorbitan trioleate, sorbitan palmitate, sorbitan stearate, sorbitan tristearate, sorbitan oleate, and sorbitan sesquioleate. Preferred sorbitan fatty acid esters include sorbitan laurate and sorbitan trioleate. These sorbitan derivates are commercially available form ICI Americas, and are sold under the trademark SPAN® and ARLACEL®, with various alphsnumeric designations for the different derivatives. The sorbitol fatty acid esters have the added benefit of having emulsifying properties. Emulsifiers can improve the solubility of various additives in the composition, such as fragrances or coloring agents, thereby improving the clarity of the composition when such additives are present.

Mineral oil can also be used as solvent in the invention. The mineral oil can be light mineral oil or heavy mineral oil. Light mineral oil are preferred for use in the invention. Mineral oils are available commercially in both USP and NF grades. USP mineral oils have viscosity that range 35 cSt to 100 cSt and pour points that range from −40° C. to −12° C. NF light mineral oils have lower viscosity, typically 3-30 cSt, and pour points as low as −45° C.

Additional Components

One or more plasticizers may be added to the composition of the present invention. When present, the plasticizers total about 0-60% by weight of the compositon, preferably about 20-50% by weight of the composition. Plasticizers function in the present invention is to increase the structural flexibility of the composition, allowing the composition to alter its shape instead of cracking or splitting in response to the thermal stresses. Adipate esters are preferred for use as plasticizers in the invention. Examples include, but are not limited to, dioctylphthalate, bis(2-ethylhexyl) terephthalate, bis(2-ethylhexyl) adipatate and tris(2-ethylhexyl)trimelltate. Suitable plasticizers also included mixtures of these plasticizers. A dicapryl adipate is a preferred plasticizer,

The composition of the present invention may also contain one or more fragrances. The fragrances may be those fragrances suitable for use in lipsticks; such type of fragrances will be well known to those of ordinary skill in the art. Examples of such fragrances include, but are not limited to, Citronella AN114351 sweet peach, AN114349 Mountain Berry, AN 114462 Lavender Meadows from Noville Corp., South Hackensack, N.J. The amount of fragrance which should be present in the composition will depend on the intensity of the fragrance and the degree to which it is desired that the composition emit fragrance.

The composition of the present invention may also contain a coloring agent, which produces a desired color appearance. A composition having a coloring agent would preferably be transparent and provide clear color without adding any haziness or cloudiness to the composition. The coloring agent may, for example, be a pigment or a dye, however, a dye is preferred for providing color, especially oil soluble dyes. Examples of such dyes include, but are not limited to, D&C violet #2, D&C yellow #11, D&C green #6, and D&C red #17.

### EXAMPLES

Transparent lipsticks representative of the present invention were prepared as follows:

### Example 1

(1) Caprylic/capric acid triglyceride was added into a steel container, which was stirred and heated to a temperature of 110° C.

(2) The gellant Versamid 1655® was added little by little, while the content of this container was heated to about 94-96° C. and stirred. During the addition of gellant, isostearic acid, isostearic alcohol, and isopropyl palmitate was added to obtain homogeneous solution.

(3) The composition was poured into a mold. The composition was allowed to cool to room temperature and solidify.

### Example 2

(1) Caprylic/capric acid triglyceride was added into a steel container, which was stirred and heated to a temperature of 110° C.

(2) The gellant UniClear 100® was added little by little, while the content of this container was heated to about 94-96° C. and stirred. During the addition of gellant, isostearic acid, isostearic alcohol, and isopropyl palmitate was added to obtain homogeneous solution.

(3) The composition was poured into a mold. The composition was allowed to cool to room temperature and solidify.

### Example 3

General Procedure For Preparation Of Compositions Of Table 1

(1) The solvent was added into a container which was agitated and heated to Temperature 110° C.

(2) The gellant UniClear 100® were added little at time, while the content of this container was heated to 95-100° C. During the addition of gellant, Isostearic acid was added to reach the homogeneous solution.

(3) The remaining components were added individually in an order determined by the amount of the component in the composition, with the largest amount being added first. Thus, the fragrance and coloring agent were added at last.

(4) The composition was poured into a mold and the composition was allowed to cool to room temperature and solidify.

Using the above procedure, the composition listed in Table 1 were prepared

| Composition(wt%) | 1 | 2 | 3 | 4 | 5 | 6 | 7 | 8 | 9 |
|---|---|---|---|---|---|---|---|---|---|
| Gellant 1 | | | | 20 | 12.5 | | | | |
| Gellant 2 | 40 | 40 | 30 | 20 | 37.5 | | | 70 | 40 |
| Gellant 3 | | | | | | 45 | 45 | | |
| Isostearyl alcohol | 5 | | | 5 | 5 | 5 | 5 | 5 | |
| Isostearic acid | 5 | 5 | 10 | 5 | 5 | 5 | 5 | 5 | 5 |
| Isopropyl palmitate | 5 | | 10 | | 5 | 5 | 5 | 5 | |
| Caprylic/Capric acid | 45 | 55 | 50 | 45 | 35 | | | 15 | |
| triglyceride | | | | | | | | | |
| 2-Ethylhexyl | | | | | | 40 | | | |
| Stearate | | | | | | | | | |
| Dicapryl Adipate | | | | | | | 40 | | |
| Mineral oil | | | | | | | | | 55 |

Gellant 1: Versamid 1655 ®
Gellant 2: UniClear 100 ®
Gellant 3: Sylvaclear A200 ®

### Example 4

Transparency Measurements

A sample cut in a thickness of 1 cm was placed on printed characters of 12 points to examine if they can be identified through the sample.

The evaluation is as follows: O, clearly identified; ∇, barely identified; and X, unidentified.

Each of compositions 1 through 10 in Example 3 was evaluated using this method.

| Composition | Transparency |
|---|---|
| 1 | ◯ |
| 2 | ◯ |
| 3 | ◯ |
| 4 | ◯ |
| 5 | ◯ |
| 6 | ◯ |
| 7 | ◯ |
| 8 | ◯ |
| 9 | ◯ |
| 10 | ◯ |

What is claimed is:

1. A transparent lipstick comprising:
(i) a polyamide gellant, selected from the group consisting of fatty polyamide and ester terminated polyamide;
(ii) a polyol fatty acid ester solvent having a carbon chain length of fatty acid at least about 6 carbon and at least about 3 carbons in carbon chain length of polyol alcohol; and
(iii) a fatty acid solubilizer, said solubilizer comprising hydrocarbon oil in the transparent lipstick.

2. The transparent lipstick of claim 1 further comprising a fatty acid solubilizer having a carbon chain of between about 10 to about 20 carbons

3. The transparent lipstick of claim 1 further comprising a fatty alcohol solubilizer having a carbon chain of between about 10 to about 20 carbons

4. The transparent lipstick of claim 1 further comprising a fatty acid ester solubilizer having a carbon chain of between about 10 to about 20 carbons

5. The transparent lipstick of claim 1 wherein the solubilizer comprise a isostearic acid, said transparent lipstick further comprising isostearyl alcohol, or isopropyl palmitate.

6. The transparent lipstick of claim 1 wherein said polyamide gellant comprises a mixture of fatty polyamide and ester terminated polyamide

7. The transparent lipstick of claim 6 wherein the fatty polyamide is present in a weigh ratio of parts of fatty polyamide to parts polyamide gellant about 1:11 to about 3:5

8. The transparent lipstick of claim 6 wherein the fatty polyamide is present in a weigh ratio of parts of fatty polyamide to parts polyamide gellant about 1:9 to about 1:4

9. The transparent lipstick of claim 6 wherein the ester terminated polyamide is present in a weigh ratio of parts of ester terminated polyamide to parts ester polyamide gellant about 2:5 to about 10:11

10. The transparent lipstick of claim 6 wherein the ester terminated polyamide is present in a weigh ratio of parts of ester terminated polyamide to parts ester polyamide gellant about 7:10 to about 1:2

11. The transparent lipstick of claim 1 wherein the polyol fatty acid ester comprises a glycerol fatty acid ester, and a sorbitol fatty acid ester.

12. The transparent lipstick of claim 1 wherein the hydrocarbon oil comprises light mineral oil.

13. The transparent lipstick of claim 1 wherein said polyamide gellant comprises between about 15 to about 90% by weight of the article, said fatty acid ester comprises between about 2 to about 20%

14. The transparent lipstick of claim 1 further comprising one or more plasticizers

15. The transparent lipstick of claim 13 wherein the plasticizers comprise between about 0-60% by weight of the composition.

16. The transparent lipstick of claim 1 further comprising one or more fragrances.

17. The transparent lipstick of claim 1 further comprising one or more coloring agents.

18. A transparent lipstick, comprising:
About 0-30% by weight fatty polyamide;
About 15-90% by weight of ester terminated polyamide;
About 0-70% by weight of mineral oil
About 1-60% by weight of polyol fatty acid ester, said comprising a linear carbon chain of between about 6 to about 22 carbons;
About 1-25% by weight of isostearic acid;
About 0-10% by weight of isostearyl alcohol;
About 0-20% by weight of isopropyl palmitate;
About 0-5% by weight of fragrance;
About 0-5% by weight of coloring agents.

19. The transparent lipstick of claim 16 wherein the fatty polyamide comprises Vresamid 1655®.

20. The transparent lipstick of claim 16 wherein the ester terminated polyamide comprises either UniClear 100® or Sylvaclear A200®.

21. A transparent lipstick, comprising:
about 30-45% by weight of Sylvaclear A200®;
About 2-6% by weight of isostearic acid;
About 2-6% by weight of isostearyl alcohol;
About 2-6% by weight of isopropyl palmitate;
About 30-40% by weight of dicapryl adipate
About 0-5% by weight of fragrance;
About 0-5% by weight of coloring agents.
